(19) United States
(12) Patent Application Publication
(10) Pub. No.: US 2004/0219179 A1
(43) Pub. Date: Nov. 4, 2004

(54) Skin abrasive agents

(76) Inventor: David H. McDaniel, Virginia Beach, VA (US)

Correspondence Address: Wayne C. Jaeschke, Jr., Morrison & Foerster LLP, Suite 300, 1650 Tysons Boulevard, McLean, VA, 22102, US

(21) Appl. No.: 10/665,390

(22) Filed: Sep. 22, 2003

Related U.S. Application Data
(60) Provisional application No. 60/411,870, filed on Sep. 20, 2002.

(51) Int. Cl. A61K 7/00

(52) U.S. Cl. 424/401

(57) ABSTRACT

The present invention relates to compositions and methods for microderm abrasion. In particular, the invention employs micro-sized particles that may be impacted into mammalian skin. The particles may, themselves, include an active agent that is to be delivered through the stratum corneum, or an active agent may be delivered through the abraded stratum corneum. The stratum corneum may be abraded by active agents or topical compositions formed into particles, hyperbaric particles, or other means.

### FIELD OF THE INVENTION

The invention relates to new compositions and methods for carrying out microderm abrasion, a process which removes a portion of the stratum corneum to allow for the delivery of an active agent therethrough. Methods of the present invention may include the use of the active agent, having been formed into a granule, to be used as the abrasive agent, or a first agent may be used to abrade the skin and a second, active, agent may be delivered afterward.

### BACKGROUND OF THE INVENTION

Microdermabrasion is a term used to describe a wide variety of processes whereby the skin's stratum corneum (“SC”), or a portion thereof, is removed by an abrasive process. In some situations the entire stratum corneum is removed, along with a portion of the epidermal layer, such that pin point bleeding of the skin may occur. However, the invention is typically carried out by removing the stratum corneum, or a portion thereof.

The stratum corneum acts as a protective or barrier layer for the skin. Removing all or a portion of the stratum corneum typically diminishes this barrier's function and thus increases the permeability of the skin to agents which can migrate through the remaining barrier. Some molecules, however, are too large in diameter to penetrate the remaining barrier.

Various techniques are also well known in the art for ‘enhancing penetration’ of the skin. These techniques can be made more effective or more efficient when combined with microdermabrasion and its variants such as chemical and laser peels or other modalities such as electrical stimulation, iontophoresis, etc.

One method of carrying out microdermabrasion is to direct a stream of abrasive particles at the skin surface using air pressure. Such air pressure may be either positive or negative or combinations of both. Typically a handpiece delivery device delivers a flow of abrasive particles and the pressure, angle of attack, hardness of the particles, diameter of particles, shape of particles and many other factors, especially the velocity and kinetic energy of the particles, affects the amount of stratum corneum removed from the skin. The duration or time of contact/exposure and the number of repetitive passes of the handpiece over the skin also affect the stratum corneum removal.

There are other abrasive devices and systems in practice as well. These range from utilizing current hardware grade abrasive paper or sandpaper which has been sterilized and hand held or used with some structurally supporting backing to facilitate the abrasion. Other methods use vibration, oscillation, spinning, and/or other vibratory or mechanical (non air driven) devices for removing a portion or all of the stratum corneum. These devices may be mechanically driven by motors or other devices or they may be ‘manual’ systems using human hands, such as hand-held microderm abraders which typically have an array of micron-sized, knife-like blades which are used to cut through the upper epithelial layers, i.e., the stratum corneum, to facilitate the transmission of compositions through the stratum corneum and into the lower epithelial layers or bloodstream of the patient. Disposable or reusable pads or bars or discs or other devices common in the art are used to provide contact between the skin and the abrasive material. Thus, there are many ways to apply an abrasive material to another material for these versions of microdermabrasion—some of which do not utilize loose abrasive powder as is common with the air abrasive devices. An abrasive may be added to a skin cream or lotion or similar product as well.

In current practice, the primary goal of the abrasive particles is abrasion of the skin. Aluminum oxide (corundum) is the most commonly used abrasive. However, sodium chloride salt abrasion is also used and sodium bicarbonate and other materials also have been and are being utilized. Since there is both tissue exposure and also some potential aerosolization of small particles in the micron-diameter range, some concern about systemic absorption, toxicity issues, and also possible pulmonary hazards from ‘abrasive dust’ have been hotly debated.

The use of microdermabrasion as a treatment before or after the application of ultrasound has been described as both an adjunctive cosmetic treatment but also as a means of enhancing delivery of topical agents both into the epidermis or dermis of the skin (and primarily confined to that target region) or alternatively to deliver a topically applied agent transdermally into deeper tissues or even for intentional systemic absorption (for example one might use this process for ‘needleless’ delivery of daily insulin medication). Illustrative of the use of ultrasound for enhancing the penetration of a topical agent through the skin are U.S. Pat. No. 6,398,753 (McDaniel) and U.S. Pat. No. 6,030,374 (McDaniel), both entitled are “Ultrasound Enhancement of Percutaneous Drug Absorption”, both of which are hereby incorporated by reference in their entireties.

### SUMMARY OF THE INVENTION

The devise of the present invention includes a motor, an oscillator driven by the motor, and an abrader pad attached to the oscillator, whereby turning on the motor and contacting the abrader pad to mammalian skin effects microderm abrasion. Further, the abrader pad may include a topical composition disposed thereon. The topical composition may be selected from the group consisting of naturally occurring chlorophyll-containing compounds, carotenoid-containing compounds, phyocobilin compounds, indocyanine green, methylene blue, rose Bengal, Vitamin C, Vitamin E, Vitamin D, Vitamin A, Vitamin K, Vitamin F, Retin A (Tretinoin), Adapalene, Retinol, Hydroquinone, Kojic acid, a growth factor, echinacea, an antibiotic, an antifungal, an antiviral, a bleaching agent, an alpha hydroxy acid, a beta hydroxy acid, salicylic acid, antioxidant triad compound, a seaweed derivative, a salt water derivative, algae, an antioxidant, a phytoanthocyanin, a phytonutrient, plankton, a botanical product, a herbaceous product, a hormone, an enzyme, a mineral, a cofactor, an antiaging substance, insulin, minoxidil, lycopene, a natural or synthetic melanin, a metalloproteinase inhibitor, proline, hydroxyproline, an anesthetic, chlorophyll, bacteriochlorophyll, copper chlorophyllin, chloroplasts, carotenoids, phycobilin, rhodopsin, anthocyanin, inhibitors of ornithine decarboxylase, inhibitors of vascular endothelial growth factor (VEGF), inhibitors of phospholipase A2, inhibitors of S—adenosylmethionine, licorice, licochalone A, genestein, soy isoflavones, phtyoestrogens, derivative, analogs, homologs, and subcomponents thereof, and derivatives, subcomponents, immunological complexes and antibodies of said target tissue, and synthetic and natural analogs thereof, and combinations thereof.

The method of the present invention may include forming a topical composition into a granule; and abrading mammalian skin by impacting the mammalian skin with the granules. In one preferred embodiment, the granules are micron-sized particles. Alternatively, the granules have a size of from about 1 to about 500 microns, about 50 to about 400 microns, about 100 to about 250 microns, or from about 1 to about 5 microns. In another embodiment, it may be preferable for the granules to have a size approximately equal to the size of the pores of the mammalian skin.

In another embodiment of the method, the granules are impacted against the mammalian skin by a pressurized gas. In this or other embodiments of the invention, the granules may comprise a topical composition disposed within or upon a secondary substance. Such a secondary substance may be selected from the group consisting of microsponges, nanodevices, liposomes, and combinations thereof. Alternatively, the granules may be hyperbaric particles. As well, the granules may be impacted against the mammalian skin, while under a vacuum.

Topical compositions exemplary of those which may be used in accordance with the present method can be selected from the group consisting of naturally occurring chlorophyll-containing compounds, carotenoid-containing compounds, phyocobilin compounds, indocyanine green, methylene blue, rose Bengal, Vitamin C, Vitamin E, Vitamin D, Vitamin A, Vitamin K, Vitamin F. Retin A (Tretinoin), Adapalene, Retinol, Hydroquinone, Kojic acid, a growth factor, echinacea, an antibiotic, an antifungal, an antiviral, a bleaching agent, an alpha hydroxy acid, a beta hydroxy acid, salicylic acid, antioxidant triad compound, a seaweed derivative, a salt water derivative, algae, an antioxidant, a phytoanthocyanin, a phytonutrient, plankton, a botanical product, a herbaceous product, a hormone, an enzyme, a mineral, a cofactor, an antiaging substance, insulin, minoxidil, lycopene, a natural or synthetic melanin, a metalloproteinase inhibitor, proline, hydroxyproline, an anesthetic, chlorophyll, bacteriochlorophyll, copper chlorophyllin, chloroplasts, carotenoids, phycobilin, rhodopsin, anthocyanin, inhibitors of ornithine decarboxylase, inhibitors of vascular endothelial growth factor (VEGF), inhibitors of phospholipase A2, inhibitors of S—adenosylmethionine, licorice, licochalone A, genestein, soy isoflavones, phtyoestrogens, derivative, analogs, homologs, and subcomponents thereof, and derivatives, subcomponents, immunological complexes and antibodies of said target tissue, and synthetic and natural analogs thereof, and combinations thereof.

### BRIEF DESCRIPTION OF THE DRAWINGS

FIG. 1 illustrates the effects of various vitamin compositions on trans-epidermal water loss.

FIG. 2 is a photograph showing various vitamin compositions in dry, granular form.

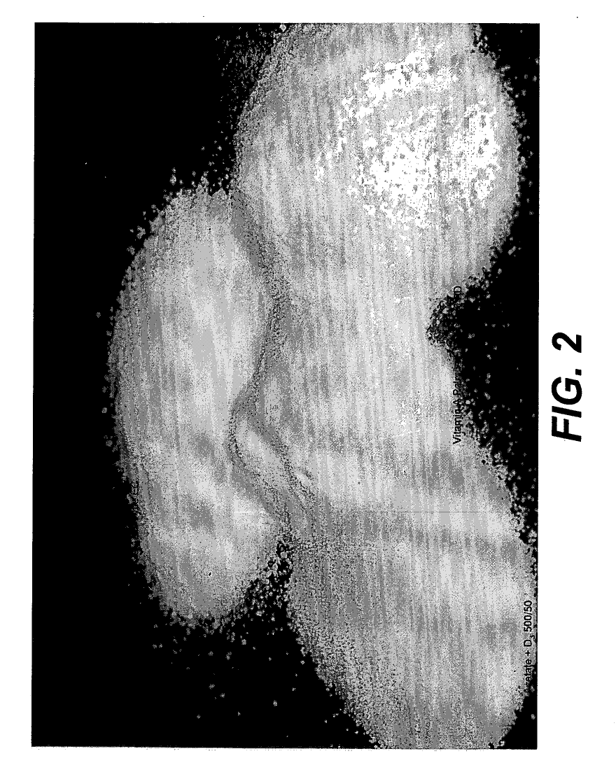

FIG. 3 is a photograph illustrating pre and post skin abrasion in conjuction with the application of a dry vitamin composition.

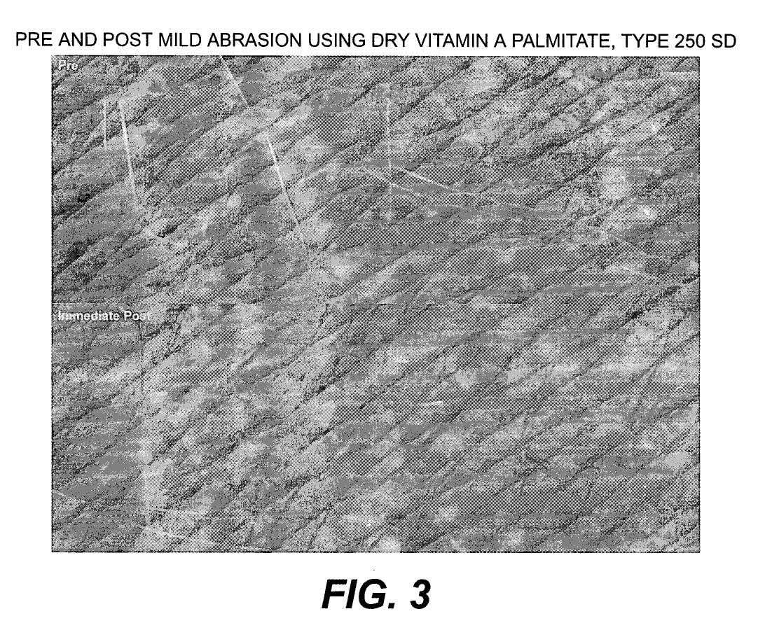

FIG. 4 is a photograph illustrating pre and post skin abrasion in conjuction with the application of another dry vitamin composition.

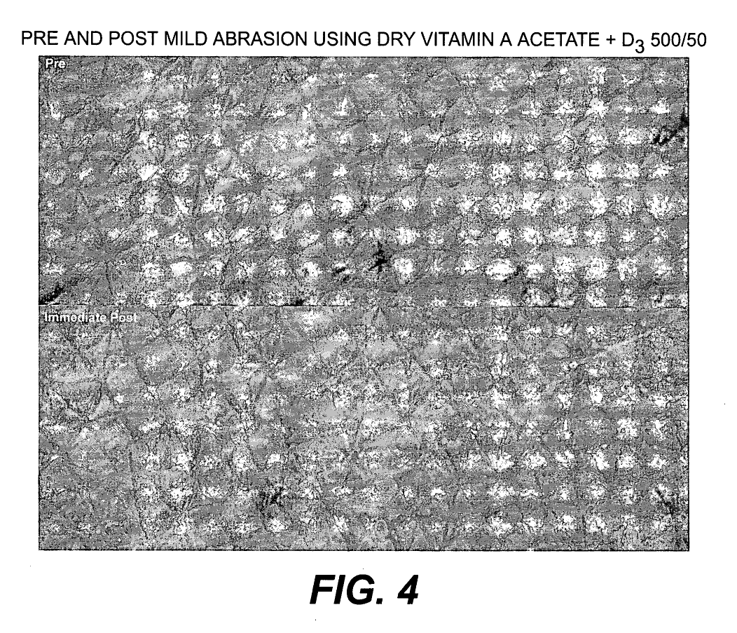

FIG. 5 is a photograph illustrating pre and post skin abrasion in conjuction with the application of another dry vitamin composition.

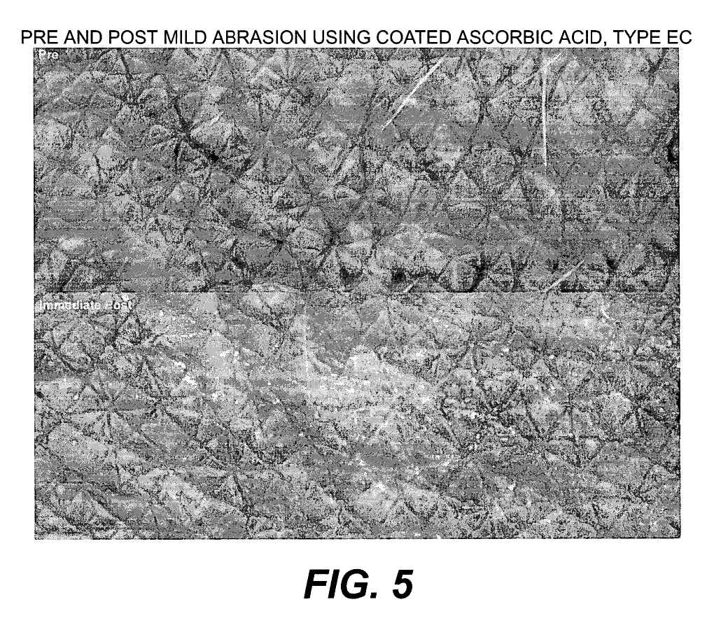

FIG. 6 is a photograph illustrating pre and post skin abrasion in conjuction with the application of another dry vitamin composition.

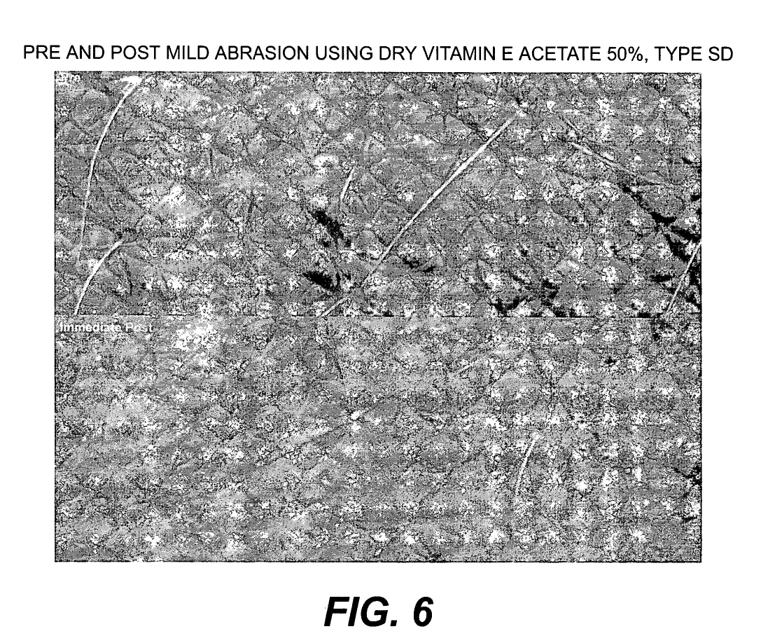

### DETAILED DESCRIPTION OF THE INVENTION

The present invention relates to new abrasive compounds to be used in microderm abrasion techniques. The agent to be delivered to the skin is a topical agent that is applied to the skin as a separate step or process from the actual skin abrasion. The abrasive functions as a mechanical abrasive and can be delivered by various methods.

One such embodiment can allow the abrasive agent to function also as the topically delivered agent. Yet another novel embodiment can use a typical abrasive agent applied first and then a second abrasive agent which is actually driven in by the abrasive device and ‘delivered’ as an ‘active agent’ using the same or a different type of abrasive device.

This invention can utilize novel compounds formed into an ‘abrasive’ form which serve either a dual abrasive—active agent combination or which primarily function as an active agent whose ‘delivery’ into or through the skin is in some manner enhanced by being applied in an abrasive fashion. Suitable active agents for use in topical compositions applied to the skin in accordance with the present invention include one or more of Vitamin C, Vitamin E, Vitamin D, Vitamin A, Vitamin K, Vitamin F, Retin A (Tretinoin), Adapalene, Retinol, Hydroquinone, Kojic acid, a growth factor, echinacea, an antibiotic, an antifungal, an antiviral, a bleaching agent, an alpha hydroxy acid, a beta hydroxy acid, salicylic acid, antioxidant triad compound, a seaweed derivative, a salt water derivative, algae, an antioxidant, a phytoanthocyanin, a phytonutrient, plankton, a botanical product, a herbaceous product, a hormone, an enzyme, a mineral, a genetically engineered substance, a cofactor, a catalyst, an antiaging substance, insulin, trace elements (including ionic calcium, magnesium, etc), minerals, minoxidil, a dye, a natural or synthetic melanin, a metalloproteinase inhibitor, proline, hydroxyproline, an anesthetic substance, chlorophyll, bacteriochlorophyll, copper chlorophyllin, chloroplasts, carotenoids, phycobilin, rhodopsin, anthocyanin, and derivatives, subcomponents, immunological complexes and antibodies directed towards any component of the target skin structure or apparatus, and analogs of the above items both natural and synthetic, as well as combinations thereof.

Preferred among compositions which may be prepared in suitable form for mechanical microderm abrasion are those selected from naturally occurring chlorophyll-containing compounds, carotenoid-containing compounds, phyocobilin compounds, indocyanine green, methylene blue, rose Bengal, Vitamin C, Vitamin E, Vitamin D, Vitamin A, Vitamin K, Vitamin F, Retin A (Tretinoin), Adapalene, Retinol, Hydroquinone, Kojic acid, a growth factor, echinacea, an antibiotic, an antifungal, an antiviral, a bleaching agent, an alpha hydroxy acid, a beta hydroxy acid, salicylic acid, antioxidant triad compound, a seaweed derivative, a salt water derivative, algae, an antioxidant, a phytoanthocyanin, a phytonutrient, plankton, a botanical product, a herbaceous product, a hormone, an enzyme, a mineral, a cofactor, an antiaging substance, insulin, minoxidil, lycopene, a natural or synthetic melanin, a metalloproteinase inhibitor, proline, hydroxyproline, an anesthetic, chlorophyll, bacteriochlorophyll, copper chlorophyllin, chloroplasts, carotenoids, phycobilin, rhodopsin, anthocyanin, inhibitors of ornithine decarboxylase, inhibitors of vascular endothelial growth factor (VEGF), inhibitors of phospholipase A2, inhibitors of S—adenosylmethionine, licorice, licochalone A, genestein, soy isoflavones, phtyoestrogens, derivative, analogs, homologs, and subcomponents thereof, and derivatives, subcomponents, immunological complexes and antibodies of said target tissue, and synthetic and natural analogs thereof, and combinations thereof.

These agents can be either formed into an abrasive substance by virtue of their crystalline phase and selected for diameter if needed for uniform flow through air abrasive devices (to minimize clogging and maximize particle velocity and flow rate). They can also be attached to various structures via adhesives (for example small sanding disc like constructions). They can be in a topical agent such as a lotion or on a solid bar, etc. Some agents which cannot be formed into such structures may alternatively be placed into synthetic spheres such as microsponges. Nanodelivery devices may be used. Liposomes and other organic delivery devices are possible for some applications. Even gases such as oxygen properly configured into small ‘hyperbaric particles’ may be utilized in this technique.

One example of this invention is the use of uniform diameter micron range particle of a vitamin A substance such as retinyl palmitate. These particles are currently available in such a standardized format for use in such applications as animal feed additives and the stability and safety data are already well documented. Similar products are available for vitamin C (ascorbic acid) and vitamin E (tocopherol acetate) and there are a myriad of forms of similar products which naturally occur in crystalline forms and which can be utilized for this invention. The hardness of such forms varies widely and many do not produce the same degree of abrasion of the stratum corneum as do current particles such as corundum. However some are sufficiently hard to allow their use a both the primary abrasive agent and also during the process be the active agent for delivery into or through the skin.

Other such agents need to either be mixed with a more effective and harder abrasive or they need to follow in a second stage or step the use of such a more effective abrasive agent. The topical crystalline vitamins above are examples of agents which can be used either as primary abrasives or as co abrasives or as secondary abrasives used following the primary abrasive.

Most microdermabrasion and other abrasive or peeling procedures are performed at intervals of weeks or months to allow recovery and repair of the disrupted epidermal or stratum corneum barrier function of the skin. This repair process in itself affects skin lipids and other cell signaling systems are activated and thus the dermal layer of the skin can be indirectly altered. The most common alteration is the production of new collagen and other extracellular matrix (ECM) materials and ground substance, etc. This is considered beneficial for anti-aging and other skin therapies. The use of topical vitamin A, C and E individually or in various combinations is well documented and produces desirable effects on the skin—including the dermal collagen and ECM stimulation.

The use of this invention provides a new and novel method of simultaneously or sequentially stimulating the ECM while performing microdermabrasion. The ‘softer’ abrasive forms of these agents can be used to allow more frequent applications of skin abrasion—for example a milder vitamin crystal abrasive can be used as a daily or weekly or even multiple times daily abrasive scrub, or perhaps in individual disposable abrasive cloths or pads. Various devices can be used to deliver these forms of abrasive vitamin agents including vibration, mechanical, and air abrasive positive or negative or combined devices. The vitamins can be adhered, impregnated or attached in many ways to construct an abrasive matrix or device for this invention.

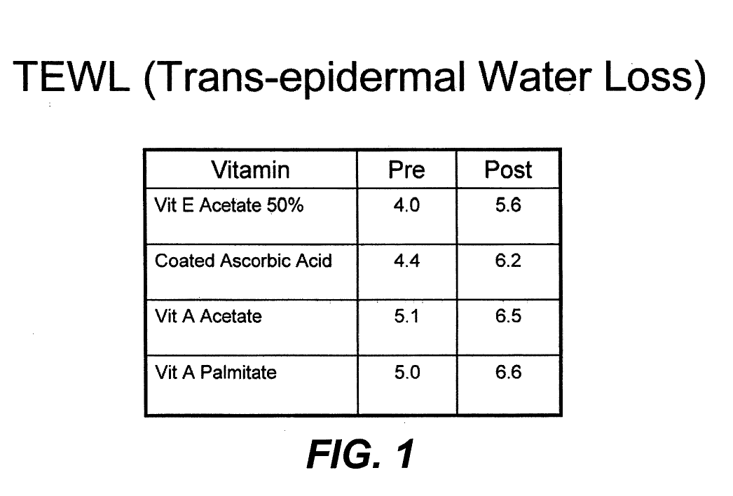

Laboratory data used to study skin surface abrasion, removal of the stratum cornum and epidermal barrier disruption commonly include, among many methods, the use of Trans Epidermal Water Loss (TEWL), skin moisture measurements (the barrier disruption also allows moisture within the skin to ‘leak out’ and thus skin surface moisture can be an effective indirect indication of barrier disruption, skin microscopy, close up photography, digital skin profilometry to map surface ‘terrain’ changes from the abrasion, skin conductance, and other measurements. We examined the various measurements listed above with various topical agents including the vitamins previously described and found varying amounts of abrasion of the stratum corneum, as shown in FIG. 1. Typically, the harder the compound the more effective the abrasion is, when all other factors are controlled.

This invention can be used to deliver a wide variety of topical agents which are biologically active in the skin of humans and animals by means of direct abrasion or secondary or co-abrasion (using other abrasive agents) utilizing a wide variety of skin abrasion and microdermabrasion devices and techniques. Thus the invention can function either as an abrasive agent, a biologically active agent, or various combinations of these two applications. Active agents whose physical chemical properties make them unsuitable in any form as abrasive agents, may nonetheless be incorporated into various ‘delivery vehicles or agents’ which do have abrasive physical properties and which then either transport or deliver or release the active agent.

I claim:

1. A method for microderm abrasion, comprising:
forming a topical composition into a granule; and
abrading mammalian skin by impacting the mammalian skin with the granules.

2. The method of claim 1 wherein the granules are micron-sized particles.

3. The method of claim 2, wherein the granules have a size of from about 1 to about 500 microns.

4. The method of claim 3, wherein the granules have a size of from about 50 to about 400 microns.

5. The method of claim 4, wherein the granules have a size of from about 100 to about 250 microns.

6. The method of claim 1, wherein the granules have a size of from about 1 to about 5 microns.

7. The method of claim 1, wherein the granules have a size approximately equal to the size of the pores of the mammalian skin.

8. The method of claim 1, wherein the granules are impacted against the mammalian skin by a pressurized gas.

9. The method of claim 1 wherein the granules comprise a topical composition disposed within or upon a secondary substance.

10. The method of claim 9, wherein the secondary substance is selected from the group consisting of microsponges, nanodevices, liposomes, and combinations thereof.

11. The method of claim 1 wherein the granules are hyperbaric particles.

12. The method of claim 1, wherein the granules are impacted against the mammalian skin, while under a vacuum.

13. The method of claim 1, wherein the topical composition is selected from the group consisting of naturally occurring chlorophyll-containing compounds, carotenoid-containing compounds, phyocobilin compounds, indocyanine green, methylene blue, rose Bengal, Vitamin C, Vitamin E, Vitamin D, Vitamin A, Vitamin K, Vitamin F, Retin A (Tretinoin), Adapalene, Retinol, Hydroquinone, Kojic acid, a growth factor, echinacea, an antibiotic, an antifungal, an antiviral, a bleaching agent, an alpha hydroxy acid, a beta hydroxy acid, salicylic acid, antioxidant triad compound, a seaweed derivative, a salt water derivative, algae, an antioxidant, a phytoanthocyanin, a phytonutrient, plankton, a botanical product, a herbaceous product, a hormone, an enzyme, a mineral, a cofactor, an antiaging substance, insulin, minoxidil, lycopene, a natural or synthetic melanin, a metalloproteinase inhibitor, proline, hydroxyproline, an anesthetic, chlorophyll, bacteriochlorophyll, copper chlorophyllin, chloroplasts, carotenoids, phycobilin, rhodopsin, anthocyanin, inhibitors of ornithine decarboxylase, inhibitors of vascular endothelial growth factor (VEGF), inhibitors of phospholipase A2, inhibitors of S—adenosylmethionine, licorice, licochalone A, genestein, soy isoflavones, phtyoestrogens, derivative, analogs, homologs, and subcomponents thereof, and derivatives, subcomponents, immunological complexes and antibodies of said target tissue, and synthetic and natural analogs thereof, and combinations thereof.

14. A device for performing microderm abrasion comprising a motor, an oscillator driven by the motor, and an abrader pad attached to the oscillator, whereby turning on the motor and contacting the abrader pad to mammalian skin effects microderm abrasion.

15. The device of claim 14 wherein the abrader pad includes a topical composition disposed thereon.

16. The device of claim 15 wherein the topical composition is selected from the group consisting of naturally occurring chlorophyll-containing compounds, carotenoid-containing compounds, phyocobilin compounds, indocyanine green, methylene blue, rose Bengal, Vitamin C, Vitamin E, Vitamin D, Vitamin A, Vitamin K, Vitamin F, Retin A (Tretinoin), Adapalene, Retinol, Hydroquinone, Kojic acid, a growth factor, echinacea, an antibiotic, an antifungal, an antiviral, a bleaching agent, an alpha hydroxy acid, a beta hydroxy acid, salicylic acid, antioxidant triad compound, a seaweed derivative, a salt water derivative, algae, an antioxidant, a phytoanthocyanin, a phytonutrient, plankton, a botanical product, a herbaceous product, a hormone, an enzyme, a mineral, a cofactor, an antiaging substance, insulin, minoxidil, lycopene, a natural or synthetic melanin, a metalloproteinase inhibitor, proline, hydroxyproline, an anesthetic, chlorophyll, bacteriochlorophyll, copper chlorophyllin, chloroplasts, carotenoids, phycobilin, rhodopsin, anthocyanin, inhibitors of omithine decarboxylase, inhibitors of vascular endothelial growth factor (VEGF), inhibitors of phospholipase A2, inhibitors of S—adenosylmethionine, licorice, licochalone A, genestein, soy isoflavones, phtyoestrogens, derivative, analogs, homologs, and subcomponents thereof, and derivatives, subcomponents, immunological complexes and antibodies of said target tissue, and synthetic and natural analogs thereof, and combinations thereof.
